(19) United States
(12) Patent Application Publication
(10) Pub. No.: US 2018/0333565 A1
(43) Pub. Date: Nov. 22, 2018

(54) PRESSURE WAVE DRUG DELIVERY

(71) Applicant: Moshe Ein-Gal, Ramat Hasharon (IL)

(72) Inventor: Moshe Ein-Gal, Ramat Hasharon (IL)

(21) Appl. No.: 15/600,859

(22) Filed: May 22, 2017

(51) Int. Cl. A61M 31/00; A61M 37/00

(52) U.S. Cl. CPC A61M 31/00; A61M 2210/1475; A61M 2210/1085; A61M 37/00

(57) ABSTRACT

A system and method for drug delivery includes a pressure wave generator, and a substrate which receives a pressure wave generated by the pressure wave generator. A substance is in contact with the substrate and is propelled by the substrate into a tissue.

### FIELD OF THE INVENTION

The present invention relates to drug delivery, and particularly to a method and a system for drug delivery enhanced by pressure waves over an area through which the drug is delivered.

### BACKGROUND OF THE INVENTION

The stratum corneum is the primary barrier to transdermal delivery of drugs or other molecules into the body. In contrast, the mucous membrane lacks a stratum corneum. However, even without the stratum corneum, it is not straightforward to deliver drugs into or through a biological membrane, such as but not limited to, the epithelial layer and connective tissue layer of mucous membranes.

Drug delivery across a biological membrane can be increased by changing either the resistance (the diffusion coefficient) or the driving force (the diffusion gradient). One method involves the use of penetration or chemical enhancers.

Another method of increasing the permeability of skin to drugs is iontophoresis, in which an external electric field is applied to cause ion transport or electro-osmosis of the drug.

Sonophoresis is another method in which sonic energy is used to enhance permeability of the skin and synthetic membranes to drugs and other molecules. The sonic energy is generally ultrasound at a frequency above 20 kHz.

Yet another method that has been used is microporation of the skin or mucosa. For example, U.S. Pat. No. 7,758,561 describes microporation of selected depth combined with sonic, electromagnetic, mechanical or thermal energy and/or a chemical enhancer.

### SUMMARY OF THE INVENTION

The present invention seeks to provide a novel pressure wave system and method, as is described more in detail hereinbelow, in which drug delivery is enhanced by pressure waves over an area through which the drug is delivered.

Although the invention is not limited by any theory, it is believed the method of applying pressure does not modify the drug or the membrane or skin. Instead, the application of pressure increases the rate of passive diffusion of the drug by pushing the drug molecules toward and into the tissue using pressure waves. More molecules are able to “sneak” through the existing “cracks” or pores due to increased kinetic energy.

The application of pressure increases the momentum of the drug molecules in the tissue direction independently of, and without need for modifying, the existing tissue permeability. In another embodiment, increasing the momentum of the drug molecules may be combined with increasing the tissue permeability.

The increased momentum of the drug may take place with the drug situated in a fluid medium, such as but not limited to, a liquid, spray, gel and the like.

In addition to applying pressure waves, the momentum of the drug molecules can be further increased in the direction of the tissue by directing a large area, high pressure, drug-containing fluid stream at the tissue (as opposed to a jet operable to penetrate by creating a hole).

There is provided in accordance with a non-limiting embodiment of the invention apparatus for drug delivery including a pressure wave generator, a substrate which receives a pressure wave generated by the pressure wave generator, and a substance in contact with the substrate which is propelled by the substrate into a tissue.

The substance may include a topical composition formed as a lotion, a cream, a solution, a gel, an ointment, a paste, an emulsion or a suspension.

The substrate may include a rigid membrane, a flexible membrane or an inflatable membrane.

There is provided in accordance with a non-limiting embodiment of the invention apparatus for drug delivery including a pressure wave generator and a substance contact with the pressure wave generator, the substance containing drug molecules that are propelled by the pressure waves into a tissue.

The pressure wave generator may include an electromagnetic pressure wave generator, an electrohydraulic pressure wave generator or a piezoelectric pressure wave generator.

Although the pressure wavefront area may be large, the pressure wave generator may include a focusing element for focusing pressure waves on the substrate, such as but not limited to, a reflector, a collimator or a lens.

There is provided in accordance with a non-limiting embodiment of the invention a method for drug delivery including using a pressure wave generator to generate a pressure wave and direct the pressure to a substrate, contacting a substance with the substrate and using the pressure wave to propel the substance into a tissue. The tissue may be external, located on the skin, for example, or internal, located in a bladder, a vagina, a rectum or a blood vessel, for example.

### BRIEF DESCRIPTION OF THE DRAWINGS

The present invention will be understood and appreciated more fully from the following detailed description taken in conjunction with the drawings in which:

FIGS. 1 and 2 are simplified illustrations of systems for pressure wave generation for drug delivery, constructed and operative in accordance with embodiments of the invention.

### DETAILED DESCRIPTION OF EMBODIMENTS

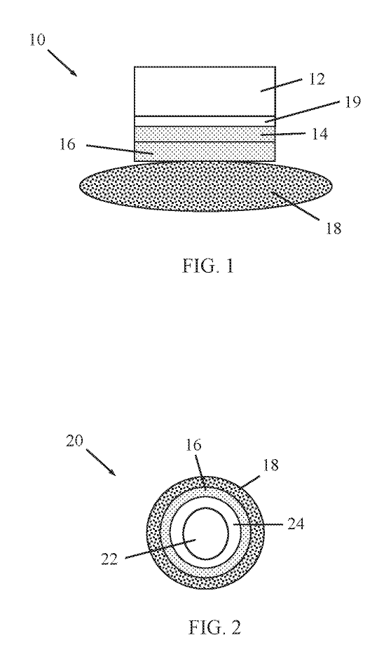

Reference is now made to FIG. 1, which illustrates a system 10 for pressure wave generation for drug delivery, constructed and operative in accordance with a non-limiting embodiment of the invention.

System 10 includes a pressure wave generator 12. A substrate 14 receives a pressure wave generated by the pressure wave generator 12. The substrate 14 may include a rigid membrane, a flexible membrane or an inflatable membrane, for example. The substrate 14 may be of any size or shape, such as but not limited to, circular, rectangular, square, or polygonal or others and any combination thereof. The substrate 14 may be planar, concave or convex and any combination thereof.

A substance 16 (any kind of drug or other medical substance, the terms being used interchangeably) may be in contact with substrate 14. The pressure wave acting on substrate 14 propels the substance 16 into a tissue 18. In another embodiment, there is no substrate 14 and the pressure wave acts on substance 16 to propel substance 16 into tissue 18.

The substance 16 may include a topical composition formed as a lotion, a cream, a solution, a gel, an ointment, a paste, an emulsion or a suspension, for example.

The pressure wave generator 12 may include an electromagnetic pressure wave generator, an electrohydraulic pressure wave generator or a piezoelectric pressure wave generator. In general, pressure wave generator 12 may include, without limitation, electromagnetic, pneumatic, piezoelectric, electrohydraulic, ballistic, pyrotechnic and others.

The pressure wave generator 12 may include a wave modifier element 19 for focusing or defocusing pressure waves impinging on the substrate 14, such as but not limited to, a reflector, a collimator or a lens.

Reference is now made to FIG. 2, which illustrates a system 20 for pressure wave generation for drug delivery, constructed and operative in accordance with a non-limiting embodiment of the invention.

Similar to the previous embodiment, system 20 includes a pressure wave generator 22. A substrate 24 receives a pressure wave generated by the pressure wave generator 22. In this embodiment, pressure wave generator 22 is configured to produce diverging waves and substrate 24 surrounds the periphery of pressure wave generator 22. As before, the pressure wave acting on substrate 24 propels the substance 16 into tissue 18. In another embodiment, there is no substrate 24 and the pressure wave acts on substance 16 to propel substance 16 into tissue 18. The tissue may be located in a bladder, a vagina, a rectum or rectum blood vessel, for example.

The systems of the invention (all embodiments) may include more than one pressure wave generator and substrate arranged in any suitable array. The array may be arranged to produce combined waves that are converging, diverging or planar. One power source may be used or a plurality of power sources, such as one power source dedicated to each pressure wave generator. The power source or sources may energize the pressure wave generators of the array at different times to produce different pressure waves and effects. The pressure wave generators of the array may be movable with respect to each other to produce different combined pressure waves and effects.

What is claimed is:

1. Apparatus for drug delivery comprising:
a pressure wave generator;
a substrate which receives a pressure wave generated by said pressure wave generator; and
a substance in contact with said substrate which is propelled by said substrate into a tissue.

2. The apparatus according to claim 1, wherein said substance comprises a topical composition formed as a lotion, a cream, a solution, a gel, an ointment, a paste, an emulsion or a suspension.

3. The apparatus according to claim 1, wherein said substrate comprises a rigid membrane.

4. The apparatus according to claim 1, wherein said substrate comprises a flexible membrane.

5. The apparatus according to claim 1, wherein said substrate comprises an inflatable membrane.

6. The apparatus according to claim 1, wherein said pressure wave generator comprises an electromagnetic pressure wave generator.

7. The apparatus according to claim 1, wherein said pressure wave generator comprises an electrohydraulic pressure wave generator.

8. The apparatus according to claim 1, wherein said pressure wave generator comprises a piezoelectric pressure wave generator.

9. The apparatus according to claim 1, wherein said pressure wave generator comprises a focusing element for focusing pressure waves on said substrate.

10. The apparatus according to claim 9, wherein said focusing element comprises a reflector, a collimator or a lens.

11. A method for drug delivery comprising:
using a pressure wave generator to generate a pressure wave and direct said pressure to a substrate;
contacting a substance with said substrate and using said pressure wave to propel said substance into a tissue.

12. The method according to claim 11, wherein said tissue is located in a bladder, a vagina or a rectum.

13. The method according to claim 11, wherein said substance comprises a topical composition formed as a lotion, a cream, a solution, a gel, an ointment, a paste, an emulsion or a suspension.
